(19) United States
(12) Patent Application Publication
(10) Pub. No.: US 2023/0393159 A1
(43) Pub. Date: Dec. 7, 2023

(54) DIMETHYLTRYPTAMINE HAPTEN, ARTIFICIAL ANTIGEN AND PREPARATION METHODS AND APPLICATION THEREOF

(71) Applicant: HANGZHOU ALLTEST BIOTECH CO., LTD, Zhejiang (CN)

(72) Inventors: Cheng LIU, Zhejiang (CN); Haijian WANG, Zhejiang (CN); Jiayi GAO, Zhejiang (CN); Fei GAO, Zhejiang (CN); Weike LU, Zhejiang (CN)

(73) Assignee: HANGZHOU ALLTEST BIOTECH CO., LTD, Zhejiang (CN)

(21) Appl. No.: 18/360,822

(22) Filed: Jul. 28, 2023

Related U.S. Application Data
(63) Continuation of application No. PCT/CN2022/116739, filed on Sep. 2, 2022.

(30) Foreign Application Priority Data
Jun. 1, 2022 (CN) 202210622055.0

(51) Int. Cl. G01N 33/94; C07D 209/16; G01N 33/577; G01N 33/58; C07K 16/44

(52) U.S. Cl. CPC G01N 33/942; C07D 209/16; G01N 33/577; G01N 33/582; C07K 16/44

(57) ABSTRACT

The present invention relates to the field of detection of dimethyltryptamine. In order to solve the problems in the prior art that liquid chromatography-tandem mass spectrometry for detection of dimethyltryptamine has high requirements for instruments and operators and is difficult to popularize and low in detection speed, the present invention discloses a dimethyltryptamine hapten, an artificial antigen and preparation methods and application thereof. The dimethyltryptamine hapten is obtained by a reaction of N,N-dimethyl-5-hydroxytryptamine as a precursor and glutaric anhydride. The artificial dimethyltryptamine antigen obtained from the dimethyltryptamine hapten can be used for preparing a dimethyltryptamine monoclonal antibody and a dimethyltryptamine colloidal gold-fluorescence test paper. The test paper has high detection sensitivity, can be used for detecting dimethyltryptamine in urine, blood, saliva and hair rapidly, has low requirements for instruments, and is easy and convenient to operate and conducive to wide popularization of drug detection.

### CROSS-REFERENCE TO RELATED APPLICATION

This application is a continuation-in-part of international application of PCT application serial no. PCT/CN2022/116739, filed on Sep. 2, 2022, which claims the priority benefit of China application no. 202210622055.0, filed on Jun. 1, 2020. The entirety of each of the above-mentioned patent applications is hereby incorporated by reference herein and made a part of this specification.

### BACKGROUND OF THE INVENTION

### 1. Technical Field

The present invention relates to the field of detection of dimethyltryptamine, and particularly relates to a dimethyltryptamine hapten, an artificial antigen and preparation methods and application thereof.

### 2. Background Art

Dimethyltryptamine (DMT) is similar to neurotransmitter serotonin, 5-methoxydimethyltryptamine, bufotenine and psilocin in structure, and is a tryptamine hallucinogen. A trace amount of the DMT can be naturally produced under catalysis of tryptamine-N-transmethylase in the brain of the human body. However, specific functions of the DMT are unclear. The DMT is secreted on the 49th day of a human embryo. Some people believe that a soul is formed afterwards, and some people even believe that the DMT is produced by the pineal gland in the brain, which can adjust the reception frequency of the human brain, so as to allow humans to perceive the non-physical world. During telepathy, some witches and wizards in North America and South America will eat some herbs containing the DMT so as to enter a trance state that seems to allow them to communicate with the gods. Ayahusca, extracted from an ayahusca herb and including the DMT as a main component, is used in religious sacrifices by Indians in North America and then gradually abused due to an euphoric effect on people. Thus, the ayahusca is called a “religious hallucinogen”. In fact, the DMT is addictive, and people will have a great change in temperament after taking the DMT and will even have mental symptoms after using the DMT for a long time. In China, the DMT belongs to first class psychotropic drugs controlled by the state and is regarded as a novel drug.

For the sake of prohibition against drugs, suspected drug users are identified by law enforcement officers by detecting residues of the DMT and metabolites thereof. At present, liquid chromatography-tandem mass spectrometry (LC-MS/MS) is usually used for qualitative and quantitative analysis of the DMT and metabolites thereof in hair. For example, according to the standard of the judicial administration industry “Determination of dimethyltryptamine and 16 novel tryptamine hallucinogens and metabolites thereof in hair samples by liquid chromatography-tandem mass spectrometry” (SF/T0065-2020) issued and implemented on May 29, 2020, the liquid chromatography-tandem mass spectrometry is specified for detecting the DMT. The method has the advantages of strong separation and analysis ability, high sensitivity, and reliable and accurate results. However, a tandem mass spectrometer has a complicated structure, high requirements for temperature and humidity in the environment, and a high maintenance cost. Mass spectrometers are high-precision instruments that may be operated by specially trained technicians, and the testing speed is slow, so that wide popularization of drug detection is not facilitated. Therefore, the development of a simple, convenient and efficient method for detecting the DMT is required.

### SUMMARY OF THE INVENTION

In order to solve the problems in the prior art that liquid chromatography-tandem mass spectrometry for detection of dimethyltryptamine has high requirements for instruments and operators, is difficult to popularize, and is slow in detection speed, the present invention provides a dimethyltryptamine hapten, an artificial antigen and preparation methods thereof. The dimethyltryptamine hapten can specifically identify dimethyltryptamine, and the artificial antigen is obtained after the dimethyltryptamine hapten is bound to a carrier protein. When the dimethyltryptamine antigen is applied in a colloidal gold-fluorescence test paper, the dimethyltryptamine in urine, blood, saliva and hair can be detected rapidly, and the test paper has high detection sensitivity and good accuracy.

In order to achieve the above purposes, the following technical solutions are adopted by the present invention.

A dimethyltryptamine hapten has a molecular structural formula as shown in Formula (I),

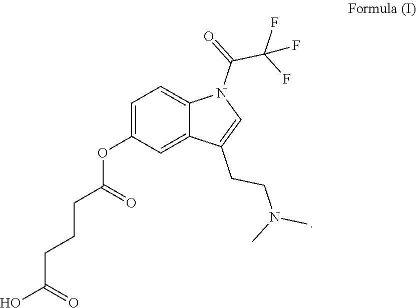 [SMILES: CN(C)CCc1cn(C(=O)C(F)(F)F)c2ccc(OC(=O)CCCC(=O)O)cc12]

The dimethyltryptamine hapten has molecular structure characteristics of the dimethyltryptamine, and the artificial antigen obtained from the hapten can be recognized by immunocompetent cells of animals to produce antibodies that can specifically bind to the dimethyltryptamine.

A preparation method of the dimethyltryptamine hapten, including the following steps:
(1) dissolving N,N-dimethyl-5-hydroxytryptamine as a precursor in benzene, adding trifluoroacetic anhydride to carry out a reaction under heating, cooling a reaction solution to room temperature after the reaction is completed, and removing the benzene to obtain a light yellow oily compound A; and
(2) dissolving the light yellow oily compound A in pyridine, adding glutaric anhydride to carry out a reaction under heating, cooling a reaction solution to room temperature after the reaction is completed, and removing the pyridine, followed by separation to obtain a yellow oily compound, namely the dimethyltryptamine hapten.

As a preference, in the step (1), the N,N-dimethyl-5-hydroxytryptamine is dissolved as a precursor in the benzene to obtain a solution with a concentration of 0.15-0.20 mmol/mL, and the solution is stirred below 0° C.; then the trifluoroacetic anhydride is slowly added, where the molar ratio of the N,N-dimethyl-5-hydroxytryptamine to the trifluoroacetic anhydride is 1:(1-1.2); the temperature is slowly raised to room temperature to carry out a reaction under stirring, and then the temperature is further raised to carry out the reaction under stirring; and after the reaction is completed, a reaction solution is cooled to room temperature, and an organic solvent is dried under reduced pressure to obtain the light yellow oily compound A.

As a better preference, in the step (1), the N,N-dimethyl-5-hydroxytryptamine is dissolved as a precursor in the benzene and then stirred below 0° C. for 5 min; then the trifluoroacetic anhydride is slowly added; the temperature is slowly raised to room temperature to carry out a reaction under stirring for 1 h, and then the reaction is carried out under stirring at 78° C. for 3 h; and after the reaction is completed, a reaction solution is cooled to room temperature, and an organic solvent is dried under reduced pressure to obtain the light yellow oily compound A.

In order to protect active sites of the dimethyltryptamine as much as possible and improve the immunogenicity and reactivity of the antigen, the N,N-dimethyl-5-hydroxytryptamine is selected as a precursor to synthesize the artificial dimethyltryptamine antigen. The structure of the N,N-dimethyl-5-hydroxytryptamine is

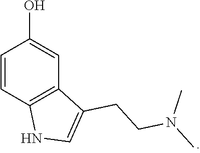

During preparation, the trifluoroacetic anhydride is used for protecting an amino group, the glutaric anhydride is used for attacking a hydroxyl at the 5th site, and a DMT derivative with a carboxyl, namely the dimethyltryptamine hapten, is obtained. The production of the light-yellow oily compound may be monitored by thin-layer chromatography during the reaction, where ethyl acetate is used as a chromatographic solution, and the Rf value of the product is 0.9.

As a preference, in the step (2), the light-yellow oily compound A is dissolved in the pyridine; the glutaric anhydride is added to carry out a reaction under heating and reflux stirring; after the reaction is completed, a reaction solution is cooled to room temperature, and a solvent is dried under reduced pressure; and then separation is performed by thin-layer chromatography to obtain the yellow oily compound, namely the dimethyltryptamine hapten.

As a better preference, in the step (2), the molar ratio of the light-yellow oily compound A to the glutaric anhydride is 1:1, and the reaction is carried out under reflux stirring at 105° C. for 18 h.

In the synthesis reaction of the dimethyltryptamine hapten, the production of the light-yellow oily compound may be monitored by thin-layer chromatography so as to determine whether the reaction is completed or not, where a chromatographic solution used includes dichloromethane, ammonia, 95% ethanol and 1,4-dioxane at a volume ratio of 10:1:8:1, and the Rf value of the product is 0.2.

An artificial dimethyltryptamine antigen is obtained by coupling the dimethyltryptamine hapten to a carrier protein and has a molecular structural formula as shown in Formula (II),

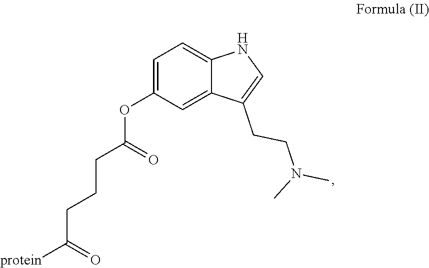

where protein refers to the carrier protein.

As a preference, the carrier protein is one of bovine serum albumin, bovine γ globulin, bovine thyroglobulin, keyhole limpet hemocyanin and ovalbumin.

A preparation method of the artificial dimethyltryptamine antigen includes the following steps:
(1) dissolving the dimethyltryptamine hapten in N,N-dimethylformamide to obtain a solution with a concentration of (0.045-0.050) mmol/mL, sequentially adding N-hydroxysuccinimide and dicyclohexylcarbodiimide to carry out a reaction under stirring at room temperature, and performing centrifugation to obtain a supernatant recorded as a solution A, where the molar ratio of the dimethyltryptamine hapten to the dicyclohexylcarbodiimide to the N-hydroxysuccinimide is 1:(1-1.2):(1.1-1.3);
(2) dissolving the carrier protein in a PBS buffer to obtain a solution B;
(3) slowly adding the solution A dropwise into the solution B under stirring, followed by placement and storage overnight to obtain an artificial antigen mixed solution; and
(4) placing the artificial antigen mixed solution in a dialysis bag for dialysis in an alkaline dialysis solution for 2-3 times, and then transferring the artificial antigen mixed solution to the PBS buffer for dialysis for 6-7 times, where the dialysis is performed for more than 2 h each time; and after the dialysis is completed, performing centrifugation to obtain a supernatant, namely the artificial dimethyltryptamine antigen, where the PBS buffer has a concentration of 0.1 mol/L and a pH value of 7.2-7.4, and the alkaline dialysis solution is a sodium carbonate solution with a pH value of 11.95-12.05.

As a better preference, in the step (1), the reaction is carried out under stirring at room temperature for 15 h.

As a better preference, in the step (2), the concentration of the carrier protein in the solution B is 5 mg/mL, and the volume ratio of the solution A to the solution B is 1:10.

As a better preference, in the step (4), the artificial antigen mixed solution is subjected to dialysis in the alkaline dialysis solution for 24 h each time.

The dimethyltryptamine hapten reacts with the N-hydroxysuccinimide and the N,N-dicyclohexylcarbodiimide to obtain an active ester, and the active ester is coupled to the carrier protein to obtain the artificial dimethyltryptamine antigen. During the reaction, active sites of the dimethyltryptamine are protected, so that the immunogenicity and reactivity of the antigen are improved. Corresponding antibodies are easily obtained during animal immunization, so that a guarantee is provided for subsequent preparation of a test reagent. Whether the hapten is successfully coupled to BSA may be determined by ultraviolet scanning of the supernatant obtained by the centrifugation after the dialysis is completed.

Application of the artificial dimethyltryptamine antigen in preparation of a dimethyltryptamine monoclonal antibody is provided.

As a preference, preparation of the dimethyltryptamine monoclonal antibody includes the following steps: diluting the artificial dimethyltryptamine antigen to 1-1.2 mg/mL with a PBS buffer, mixing the diluted artificial dimethyltryptamine antigen with an immune adjuvant at a volume ratio of 1:1-1.2 to obtain a mixture, and subcutaneously injecting the mixture into mice to repeatedly immunize the mice every 2-3 weeks; isolating serum, detecting the valence of an antibody in the serum by indirect ELISA, and when the valence of the antibody is smaller than 1:128, collecting the serum, followed by purification to obtain a polyclonal antibody; and then subjecting myeloma cells of the immunized mice to fusion with spleen B cells under the action of polyethylene glycol as a fusion promoter, screening hybridoma cells by a monoclonal cell technology, intraperitoneally injecting and inoculating the hybridoma cells into the mice, and collecting ascites 2 weeks later to obtain the dimethyltryptamine monoclonal antibody.

Application of the artificial dimethyltryptamine antigen in preparation of a dimethyltryptamine colloidal gold-fluorescence test paper.

As a preference, a test line is obtained by dotting an artificial dimethyltryptamine antigen as a raw material, a quality control line is obtained by dotting goat anti-mouse IgG or goat anti-rabbit IgG as a raw material, and a binding pad is obtained by spraying a dimethyltryptamine monoclonal antibody-colloidal gold complex and a dimethyltryptamine monoclonal antibody-fluorescein complex on the binding pad.

The dimethyltryptamine colloidal gold-fluorescence test paper of the present invention has high detection sensitivity, and the detectable concentration can reach 1,000 ng/mL. As a competition method is used as a test principle of the dimethyltryptamine test reagent strip of the present invention, the color development intensity of the test line is negatively correlated with the concentration of the dimethyltryptamine in a sample. As colloidal gold and fluorescence are used for labeling at the same time, qualitative detection and quantitative detection may be performed simultaneously. The aggregation and color development of the colloidal gold on an NC film are observed by a user with naked eyes. In a case that the quality control line occurs, a negative result is achieved when the T line occurs, and a positive result is achieved when the T line does not occur. When quantitative detection is required, a fluorescence immunity analyzer may be used for quantitative analysis.

As a preference, the quality control line and the test line are prepared in the following processes: spraying 1.0 mg/mL of goat anti-rabbit IgG and 0.1 mg/mL of goat anti-mouse IgG on a nitrocellulose membrane at a spraying rate of 1.0 μL/cm to serve as the quality control line, and spraying 0.2 mg/mL of a dimethyltryptamine antigen on the nitrocellulose membrane at a spraying rate of 1.0 μL/cm to serve as the test line.

As a preference, preparation of the dimethyltryptamine monoclonal antibody-colloidal gold complex includes the following steps: adjusting the pH value of a colloidal gold solution to 7.6, adding a dimethyltryptamine monoclonal antibody solution for stirring, and adding a 1% PEG2000 buffer to obtain a mixed solution; subjecting the mixed solution to centrifugation at 100,000 g at 4° C. for 60 min, removing a precipitate, and performing resuspension with a PEG2000 buffer repeatedly for 2-3 times to obtain a colloidal gold labeled protein; and then subjecting the colloidal gold labeled protein to purification by a gel filtration method, followed by elution with a PBS buffer containing BSA and sodium azide, and collecting the dimethyltryptamine monoclonal antibody-colloidal gold complex.

As a preference, preparation of the dimethyltryptamine monoclonal antibody-fluorescein complex includes the following steps: diluting a dimethyltryptamine monoclonal antibody to 10 mg/mL with 0.025 mol/L of CB with a pH value of 9.0, placing the dimethyltryptamine monoclonal antibody in a dialysis bag, and immersing the dialysis bag in an Alexa Fluor® dye solution, where the Alexa Fluor® dye solution is a solution with a concentration of 5 μg/mL obtained by dissolving an Alexa Fluor® dye in a PBS buffer, and the volume of the Alexa Fluor® dye solution is 10 times higher than that of the antibody solution; and performing binding under slow stirring by an electromagnetic stirrer at 4° C. for 18-24 h, taking out the dialysis bag to complete a labeling process, and subjecting a bound compound in the dialysis bag to chromatography with a Sephadex G-25 or Sephadex G-50 column to obtain the dimethyltryptamine monoclonal antibody-fluorescein complex.

As a preference, preparation of the binding pad includes the following steps: diluting a dimethyltryptamine monoclonal antibody-colloidal gold complex in a PBS buffer containing bovine serum albumin until is 1/4-1/3 of the initial OD value of the complex, mixing the diluted complex with a dimethyltryptamine monoclonal antibody-fluorescein complex at a ratio of 3:1 to obtain a mixture, and then uniformly spraying the mixture on the binding pad at a spray rate of 1.0 L/cm.

As a preference, the preparation of the binding pad further includes adding 50 mg/mL of trehalose and 200 mg/mL of sucrose into the PBS buffer containing the bovine serum albumin.

Through addition of the sucrose and the trehalose, the activity of the dimethyltryptamine monoclonal antibody-colloidal gold complex is effectively maintained, the validity period of the binding pad is prolonged, and properties of a product are improved.

Therefore, the present invention has the following beneficial effects. (1) The dimethyltryptamine haptene and the artificial dimethyltryptamine antigen are synthesized by using the N,N-dimethyl-5-hydroxytryptamine as a precursor, so that active sites of the dimethyltryptamine are not destroyed in the preparation process, and the obtained antigen has good immunogenicity and reactivity. Corresponding antibodies can be easily obtained during animal immunization and used for preparing a dimethyltryptamine colloidal gold-fluorescence test paper. (2) The dimethyltryptamine colloidal gold-fluorescence test paper provided by the present invention has high sensitivity, the detection limit of the colloidal gold is 1,000 ng/mL, and the color development intensity of the test line is negatively correlated with the concentration of the dimethyltryptamine. The dimethyltryptamine in urine, blood, saliva and hair can be detected rapidly, and the concentration of the dimethyltryptamine in a sample can be obtained by qualitative detection with naked eyes or by quantitative detection of the fluorescence intensity on the basis of on-machine detection. Compared with liquid chromatography-tandem mass spectrometry, the test paper is more convenient and fast, has low requirements for instruments, and is easy and convenient to operate and conducive to wide popularization of drug detection.

### BRIEF DESCRIPTION OF THE DRAWINGS

FIG. 1 shows a liquid chromatography spectrum of a dimethyltryptamine haptene prepared in Example 1, where: mAU refers to the milli-absorbance unit, and min refers to the time unit minute; and

FIG. 2 shows an ESI-MS spectrum of the dimethyltryptamine haptene prepared in Example 1, where: Intens. refers to signal intensity, and m/z refers to the mass-charge ratio.

### DETAILED DESCRIPTION OF THE EMBODIMENTS

The present invention is further described below in combination with specific implementation methods.

A PBS buffer used in the following embodiments is prepared by dissolving 14.5 g of disodium hydrogen phosphate dodecahydrate, 43.875 g of sodium chloride and 1.495 g of sodium dihydrogen phosphate dihydrate in double distilled water to a constant volume of 5.0 L, and has a pH value of 7.4. An alkaline dialysis solution is prepared in the following process: adjusting the pH value of a sodium carbonate aqueous solution with a mass fraction of 0.5% to 12.00 with a NaOH solution with a concentration of 2 mol/L.

### Example 1

(1) Preparation of a Dimethyltryptamine Hapten:
A. 0.98 mmol of N,N-dimethyl-5-hydroxytryptamine was added as a precursor into a 50 mL one-mouth round-bottom flask, 5 mL of benzene was added for dissolution to obtain a solution, and the solution was stirred in an ice water bath at 0° C. for 5 min. 1.08 mmol of trifluoroacetic anhydride was slowly added, the temperature was slowly raised to room temperature to carry out a reaction under stirring for 1 h, and then the reaction was carried out in an oil bath under stirring at 78° C. for 3 h and monitored by thin-layer chromatography, where ethyl acetate was used as a chromatographic solution, and the Rf value of a product was 0.9. The reaction was terminated when basically completed, and a reaction solution was cooled to room temperature and then dried under a reduced pressure of −0.1 MPa at 50° C. to obtain a light-yellow oily compound A
B. 0.94 mmol of the light-yellow oily compound A obtained in the previous step was placed in a 50 mL single-mouth round-bottomed flask, 10 mL of pyridine was added for dissolution, and 0.94 mmol of glutaric anhydride was added to obtain a mixture. The mixture was placed in an oil bath to carry out a reaction under reflux stirring at 105° C. for 18 h, and the reaction was monitored by thin-layer chromatography, where a mixture of dichloromethane, ammonia, 95% ethanol and 1,4-dioxane at a ratio of 10:1:8:1 was used as a chromatographic solution, and the Rf value of a product was 0.2. The reaction was terminated after basically completed, a reaction solution was cooled to room temperature, a solvent was dried under reduced pressure, and separation was performed by thin-layer chromatography to obtain a yellow oily compound, where anhydrous ethanol was used as a solvent and an eluent, a mixture of dichloromethane, ammonia, 95% ethanol and 1,4-dioxane at a ratio of 10:1:8:1 was used as a chromatographic solution, the Rf value of the product was 0.2, and the yellow oily compound was the dimethyltryptamine hapten.

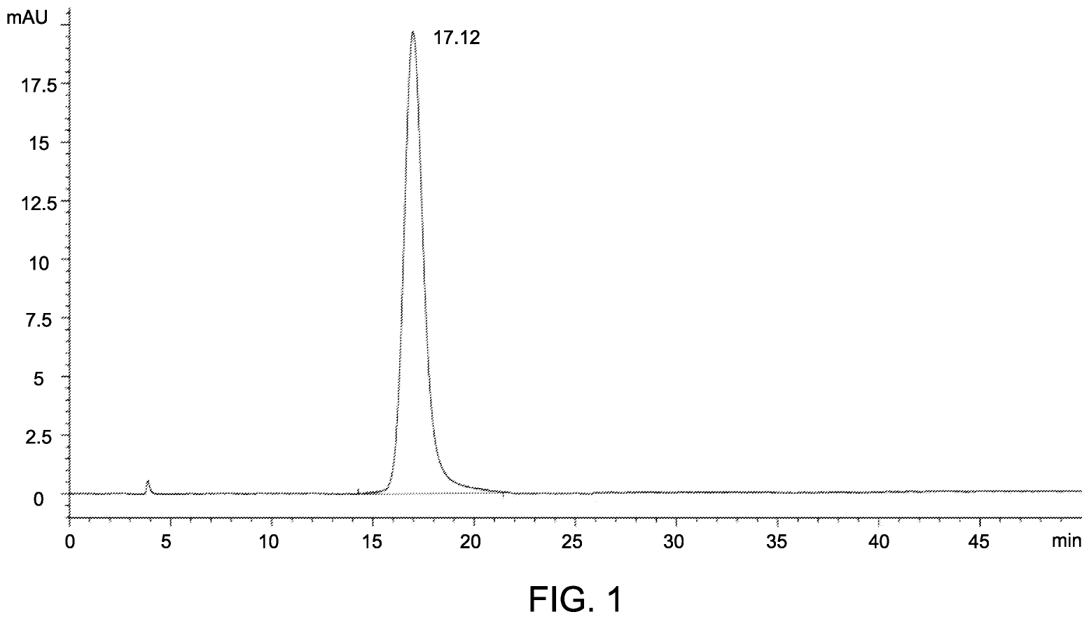

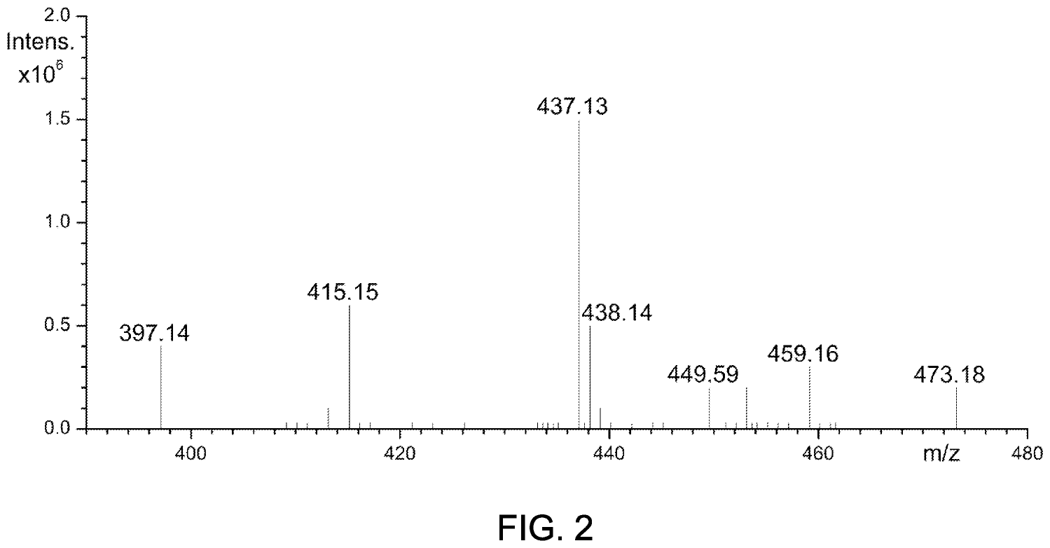

The dimethyltryptamine hapten obtained in step (1) was separately analyzed by high performance liquid chromatography (HPLC) and high resolution mass spectrometry (ESI-MS). FIG. 1 shows a liquid chromatography spectrum of an artificial dimethyltryptamine haptene I, where mAU refers to the milli-absorbance unit, and min refers to the time unit minute. From FIG. 1, it can be seen that the purity of the purified artificial dimethyltryptamine hapten I is greater than 99%, so that requirements for the preparation of an artificial antigen are completely met, and a next reaction can be carried out. FIG. 2 shows an ESI-MS spectrum of the artificial dimethyltryptamine haptene I, where Intens. refers to signal intensity, and m/z refers to the mass-charge ratio. From FIG. 2, it can be seen that the M+H molecular ion peak of the artificial dimethyltryptamine haptene I is 415.15, and the molecular mass 414.14 is consistent with the molar mass 414. Thus, it can be determined that the yellow oily compound obtained in step (1) is the artificial dimethyltryptamine haptene I designed by the present invention.

(2) Preparation of an Artificial Dimethyltryptamine Antigen:
A. 0.36 mmol of the dimethyltryptamine hapten prepared in step (1) was placed in a 25 mL single-mouth round-bottomed flask, 7.5 mL of N,N-dimethylformamide (DMF) was added, then 0.43 mmol of dicyclohexylcarbodiimide (DCC) and 0.43 mmol of N-hydroxysuccinimide (NHS) were added to carry out a reaction under stirring at room temperature for 15 h. After the reaction was completed, centrifugation was performed at 8,000 r/min and 4° C. for 15 min, and a supernatant was collected and recorded as a solution A.
B. 0.375 g of bovine serum albumin (BSA) was weighed and dissolved in 75 mL of a PBS solution to obtain a BSA solution recorded as a solution B.
C. The solution A was slowly added dropwise into the solution B at a volume ratio of 1:10 under rapid stirring to obtain a mixed solution, the obtained mixed solution was placed and stored at 4° C. overnight to obtain an immunogen and coating antigen (DMT-BSA) mixed solution, namely an artificial antigen mixed solution, and then whether the hapten was successfully coupled to the BSA was determined by ultraviolet scanning.
D. The successfully coupled artificial antigen mixed solution was transferred into a dialysis bag for dialysis in an alkaline dialysis solution for 24 h under stirring, where the dialysis was repeated for two times.
E. After the dialysis in the alkaline dialysis solution, the artificial antigen mixed solution was subjected to dialysis in a PBS solution for 7 times for 2 h each time, and after the dialysis was completed, centrifugation was performed to obtain a supernatant, namely the artificial dimethyltryptamine antigen.

### Example 2

(1) Preparation of a dimethyltryptamine hapten: The molar ratio of the N,N-dimethyl-5-hydroxytryptamine to the trifluoroacetic anhydride was 1:1, and other preparation conditions were the same as those in Example 1.
(2) Preparation of an artificial dimethyltryptamine antigen: The molar ratio of the dimethyltryptamine hapten to the DCC to the NHS was 1:1:1.1, and other preparation conditions were the same as those in Example 1.

### Example 3

(1) Preparation of a dimethyltryptamine hapten: The molar ratio of the N,N-dimethyl-5-hydroxytryptamine to the trifluoroacetic anhydride was 1:1.2, and other preparation conditions were the same as those in Example 1.
(2) Preparation of an artificial dimethyltryptamine antigen: The molar ratio of the dimethyltryptamine hapten to the DCC to the NHS was 1:1.2:1.3, and other preparation conditions were the same as those in Example 1.

### Example 4

Preparation of a Dimethyltryptamine Monoclonal Antibody:

The dimethyltryptamine antigen obtained in Example 1 was diluted to 1 mg/mL with a PBS buffer and mixed with an immune adjuvant at a volume ratio of 1:1 to obtain a mixture, and the mixture was subcutaneously injected into mice to repeatedly immunize the mice every 2 weeks. Serum was isolated, the valence of an antibody in the serum was detected by indirect ELISA, and when the valence of the antibody was smaller than 1:128, the serum was collected and purified to obtain a polyclonal antibody. Then myeloma cells of the immunized mice were fused with spleen B cells under the action of polyethylene glycol as a fusion promoter and screened by a monoclonal cell technology to obtain hybridoma cells, the hybridoma cells were intraperitoneally injected and inoculated into the mice, and ascites was collected 2 weeks later to obtain the dimethyltryptamine monoclonal antibody.

### Example 5

Preparation of a dimethyltryptamine monoclonal antibody: The dimethyltryptamine monoclonal antibody was prepared by using the dimethyltryptamine antigen obtained in Example 2, and other preparation processes were the same as those in Example 4.

### Example 6

Preparation of a dimethyltryptamine monoclonal antibody: The dimethyltryptamine monoclonal antibody was prepared by using the dimethyltryptamine antigen obtained in Example 3, and other preparation processes were the same as those in Example 4.

### Example 7

Preparation of a Dimethyltryptamine Colloidal Gold-Fluorescence Test Paper:
A. A test line and a quality control line: 0.2 mg/mL of the dimethyltryptamine antigen obtained in Example 1 was sprayed on a nitrocellulose membrane at a spraying rate of 1.0 μL/cm to serve as the test line, and 1.0 mg/mL of goat anti-rabbit IgG and 0.1 mg/mL of goat anti-mouse IgG were sprayed on the nitrocellulose membrane at a spraying rate of 1.0 μL/cm to serve as the quality control line.
B. A dimethyltryptamine monoclonal antibody-colloidal gold complex: The dimethyltryptamine monoclonal antibody obtained in Example 4 was dissolved in a colloidal gold solution to obtain a dimethyltryptamine monoclonal antibody solution, and the pH value of the colloidal gold solution was adjusted to 7.6. 1 mL of the dimethyltryptamine monoclonal antibody solution was added and stirred, and a 1% PEG2000 buffer with a volume of 10 mL was added to obtain a mixed solution. The mixed solution was subjected to centrifugation at 100,000 g at 4° C. for 60 min, a precipitate was removed, and resuspension was performed with a PEG2000 buffer repeatedly for 3 times to obtain a colloidal gold labeled protein. Then, the colloidal gold labeled protein was purified by a gel filtration method and eluted with a PBS buffer containing 1% of BSA and 0.02% of sodium azide, and the dimethyltryptamine monoclonal antibody-colloidal gold complex was collected.
C. A dimethyltryptamine monoclonal antibody-fluorescein complex: The dimethyltryptamine monoclonal antibody obtained in Example 4 was diluted to 10 mg/mL with 0.025 mol/L of CB with a pH value of 9.0 to obtain an antibody solution, and the antibody solution was placed in a dialysis bag. An opening of the dialysis bag was tightly tied with only a few gaps retained. An Alexa Fluor® dye solution with a concentration of 5 μg/mL was prepared by dissolving an Alexa Fluor® dye in a PBS buffer and then added into a beaker, where the volume of the Alexa Fluor® dye solution was 10 times higher than that of the antibody solution. The dialysis bag was immersed in the Alexa Fluor® dye solution. Binding was performed under slow stirring by an electromagnetic stirrer at 4° C. for 24 h, the dialysis bag was taken out to complete a labeling process, and a bound compound was sucked from the dialysis bag and then subjected to chromatography with a Sephadex G-25 column to remove the Alexa Fluor® dye.
D. A binding pad: The dimethyltryptamine monoclonal antibody-colloidal gold complex was diluted in a PBS buffer containing 1% of BSA, 50 mg/mL of trehalose and 200 mg/mL of sucrose until was 1/3 of the initial OD value of the complex and then uniformly sprayed on the binding pad at a spray rate of 1.0 μL/cm; and the dimethyltryptamine monoclonal antibody-fluorescein complex was uniformly sprayed on the binding pad at a spray rate of 1.0 μL/cm.
E. Assembly: A dotted sheet base plate, the binding pad, a sample pad and a water-absorbent paper were assembled into a large card and then sliced to obtain a dimethyltryptamine colloidal gold-fluorescence test paper strip.

### Example 8

Preparation of a dimethyltryptamine colloidal gold-fluorescence test paper: The dimethyltryptamine antigen obtained in Example 2 was used as a dimethyltryptamine antigen, the dimethyltryptamine monoclonal antibody obtained in Example 5 was used as a dimethyltryptamine monoclonal antibody, and other preparation processes were the same as those in Example 7.

### Example 9

Preparation of a dimethyltryptamine colloidal gold-fluorescence test paper: The dimethyltryptamine antigen obtained in Example 3 was used as a dimethyltryptamine antigen, the dimethyltryptamine monoclonal antibody obtained in Example 6 was used as a dimethyltryptamine monoclonal antibody, and other preparation processes were the same as those in Example 7.

Dimethyltryptamine solutions with different concentrations were prepared, the dimethyltryptamine colloidal gold-fluorescence test paper strip prepared in Example 8 was used for carrying out a functional test on samples, the color development intensity of the test line was determined by a colloidal gold colorimetric card, and each concentration of the sample was determined for 5 times to obtain an average value. Results are as shown in Table 1.

TABLE 1
Color development intensity of a dimethyltryptamine colloidal gold-
fluorescence test paper

| Sample concentration (ng/mL) | T line intensity | Fluorescence intensity F |
|---|---|---|
| 0 | G9 | 2301±43 |
| 250 | G6.5 | 1395±22 |
| 500 | G5 | 846±17 |
| 750 | G4 | 513±12 |
| 1000 | G3.5 | 311±13 |
| 1250 | G3 | 189±12 |
| 1500 | G2 | 114±8 |
| 1750 | G2 | 69±4 |
| 3000 | G1 | 6±1 |

Results of the colloidal gold-fluorescence test paper are read by a colorimetric card method. G1 to G10 refer to the color development degree of colloidal gold of the T line, where on the basis of observation with naked eyes, the G1 shows a colorless T line which indicates a strong positive result, and the G10 shows a dark T line which indicates a strong negative result. The fluorescence intensity of samples with different concentrations can be tested by on-machine detection. The fluorescence intensity is negatively correlated with the concentration of the dimethyltryptamine. When the concentration of the dimethyltryptamine in the samples is higher, the fluorescence intensity is lower, and an inversely proportional relationship is achieved in a certain range. From Table 1, it can be seen that the detection concentration of the test paper prepared by the present invention can be as low as 1,000 ng/mL and has high sensitivity. Results can be observed with naked eyes and can also be quantitatively analyzed by detecting the fluorescence intensity.

The dimethyltryptamine colloidal gold-fluorescence test paper strips prepared in Examples 7-9 were used for carrying out a functional test on 118 clinical urine samples. In the clinical urine samples, 73 cases were negative, and 45 cases were positive. The concentration of the dimethyltryptamine was distributed in 1,000-2,000 ng/mL. Test results are as shown in Table 2.

TABLE 2
Test results of clinical samples

| | | Clinical sample | | |
|---|---|---|---|---|
| Item | | Positive | Negative | Accuracy % |
| Example 7 | Positive | 45 cases | 3 cases | 97.5 |
| | Negative | 0 case | 70 cases | |
| Example 8 | Positive | 44 cases | 4 cases | 95.8 |
| | Negative | 1 case | 69 cases | |
| Example 9 | Positive | 41 cases | 6 cases | 92.4 |
| | Negative | 3 cases | 67 cases | |

From Table 2, it can be seen that the dimethyltryptamine colloidal gold-fluorescence test paper of the present invention has high detection accuracy for clinical samples, and the overall accuracy is greater than 90%.

What is claimed is:

1. A dimethyltryptamine hapten, wherein the dimethyltryptamine hapten has a molecular structural formula as shown in Formula (I),

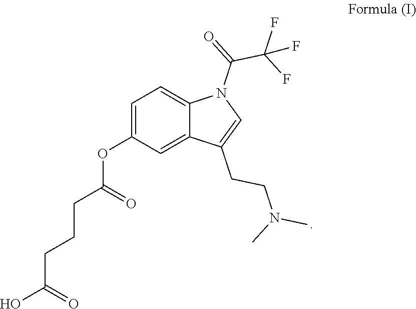 [SMILES: CN(C)CCc1cn(C(=O)C(F)(F)F)c2ccc(OC(=O)CCCC(=O)O)cc12]

2. A preparation method of the dimethyltryptamine hapten according to claim 1, comprising the following steps:
(1) dissolving N,N-dimethyl-5-hydroxytryptamine as a precursor in benzene, adding trifluoroacetic anhydride to carry out a first reaction under heating, cooling a first reaction solution to room temperature after the first reaction under heating is completed, and removing the benzene to obtain a light-yellow oily compound A; and
(2) dissolving the light-yellow oily compound A in pyridine, adding glutaric anhydride to further carry out a second reaction under heating, cooling a second reaction solution to room temperature after the second reaction under heating is completed, and removing the pyridine, followed by separation to obtain a yellow oily compound, namely the dimethyltryptamine hapten.

3. The preparation method of the dimethyltryptamine hapten according to claim 2, wherein in the step (1), the N,N-dimethyl-5-hydroxytryptamine is dissolved as a precursor in the benzene to obtain a solution with a concentration of 0.15-0.20 mmol/mL, and the solution is stirred below 0° C.; then the trifluoroacetic anhydride is slowly added, wherein a molar ratio of the N,N-dimethyl-5-hydroxytryptamine to the trifluoroacetic anhydride is 1:(1-1.2); the temperature is slowly raised to room temperature to carry out a reaction under stirring, and then the temperature is raised to carry out the reaction under stirring; and after the reaction under heating is completed, a reaction solution for the reaction under stirring is cooled to room temperature, and an organic solvent is dried under reduced pressure to obtain the light yellow oily compound A.

4. The preparation method of the dimethyltryptamine hapten according to claim 2, wherein in the step (2), the light yellow oily compound A is dissolved in the pyridine; the glutaric anhydride is added to carry out a reaction under heating and reflux stirring; after the reaction under heating and reflux stirring is completed, a reaction solution for the reaction under heating and reflux stirring is cooled to room temperature, and a solvent is dried under reduced pressure; and then separation is performed by thin-layer chromatography to obtain the yellow oily compound, namely the dimethyltryptamine hapten.

5. An artificial dimethyltryptamine antigen, wherein the artificial dimethyltryptamine antigen is obtained by coupling the dimethyltryptamine hapten according to claim 1 to a carrier protein and has a molecular structural formula as shown in Formula (II), 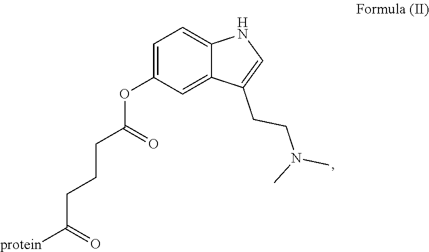
wherein in the Formula (II), protein refers to the carrier protein.

6. The artificial dimethyltryptamine antigen according to claim 5, wherein the carrier protein is one of bovine serum albumin, bovine γ globulin, bovine thyroglobulin, keyhole limpet hemocyanin and ovalbumin.

7. A preparation method of the artificial dimethyltryptamine antigen according to claim 5, comprising the following steps:
(1) dissolving the dimethyltryptamine hapten in N,N-dimethylformamide to obtain a solution with a concentration of 0.045-0.050 mmol/mL, sequentially adding N-hydroxysuccinimide and dicyclohexylcarbodiimide to carry out a reaction under stirring at room temperature, and performing centrifugation to obtain a supernatant recorded as a solution A, wherein a molar ratio of the dimethyltryptamine hapten to the dicyclohexylcarbodiimide to the N-hydroxysuccinimide is 1:(1-1.2):(1.1-1.3);
(2) dissolving the carrier protein in PBS buffer to obtain a solution B;
(3) slowly adding the solution A dropwise into the solution B under stirring, followed by placement and storage overnight to obtain an artificial antigen mixed solution; and
(4) placing the artificial antigen mixed solution in a dialysis bag for dialysis in an alkaline dialysis solution for 2-3 times, and then transferring the artificial antigen mixed solution to the PBS buffer for dialysis for 6-7 times, wherein the dialysis is performed for more than 2 h each time; and after the dialysis is completed, performing centrifugation to obtain a supernatant, namely the artificial dimethyltryptamine antigen,
wherein the PBS buffer has a concentration of 0.1 mol/L and a pH value of 7.2-7.4, and the alkaline dialysis solution is a sodium carbonate solution with a pH value of 11.95-12.05.

8. Application of the artificial dimethyltryptamine antigen according to claim 5 in preparation of a dimethyltryptamine monoclonal antibody.

9. Application of the artificial dimethyltryptamine antigen according to claim 5 in preparation of a dimethyltryptamine colloidal gold-fluorescence test paper.

10. The application of the artificial dimethyltryptamine antigen in preparation of the dimethyltryptamine colloidal gold-fluorescence test paper according to claim 9, wherein a test line is obtained by dotting the artificial dimethyltryptamine antigen as a raw material, a quality control line is obtained by dotting goat anti-mouse IgG or goat anti-rabbit IgG as a raw material, and a binding pad is obtained by spraying a dimethyltryptamine monoclonal antibody-colloidal gold complex and a dimethyltryptamine monoclonal antibody-fluorescein complex on the binding pad.

11. A preparation method of the artificial dimethyltryptamine antigen according to claim 6, comprising the following steps:
(1) dissolving the dimethyltryptamine hapten in N,N-dimethylformamide to obtain a solution with a concentration of 0.045-0.050 mmol/mL, sequentially adding N-hydroxysuccinimide and dicyclohexylcarbodiimide to carry out a reaction under stirring at room temperature, and performing centrifugation to obtain a supernatant recorded as a solution A, wherein a molar ratio of the dimethyltryptamine hapten to the dicyclohexylcarbodiimide to the N-hydroxysuccinimide is 1:(1-1.2):(1.1-1.3);
(2) dissolving the carrier protein in PBS buffer to obtain a solution B;
(3) slowly adding the solution A dropwise into the solution B under stirring, followed by placement and storage overnight to obtain an artificial antigen mixed solution; and
(4) placing the artificial antigen mixed solution in a dialysis bag for dialysis in an alkaline dialysis solution for 2-3 times, and then transferring the artificial antigen mixed solution to the PBS buffer for dialysis for 6-7 times, wherein the dialysis is performed for more than 2 h each time; and after the dialysis is completed, performing centrifugation to obtain a supernatant, namely the artificial dimethyltryptamine antigen,
wherein the PBS buffer has a concentration of 0.1 mol/L and a pH value of 7.2-7.4, and the alkaline dialysis solution is a sodium carbonate solution with a pH value of 11.95-12.05.
